(19) United States
(12) Patent Application Publication
(10) Pub. No.: US 2009/0043293 A1
(43) Pub. Date: Feb. 12, 2009

(54) MULTI-MODULE SKIN OR BODY TREATMENT DEVICE AND THE METHOD OF USING

(75) Inventors: Michail M. Pankratov, Waltham, MA (US); Nancy M. Briefs, Nashua, NH (US)

Correspondence Address: LAHIVE & COCKFIELD, LLP;FLOOR 30, SUITE 3000, ONE POST OFFICE SQUARE, BOSTON, MA, 02109, US

(73) Assignee: ELEME MEDICAL INC., Merrimack, NH (US)

(21) Appl. No.: 12/188,519

(22) Filed: Aug. 8, 2008

Related U.S. Application Data
(60) Provisional application No. 60/964,246, filed on Aug. 10, 2007.

(51) Int. Cl. A61B 18/20; A61N 7/00; A61H 23/00

(52) U.S. Cl. 606/9; 606/33; 606/131; 601/2; 601/4

(57) ABSTRACT

A skin or body treatment device includes one or more removable and replaceable modules. Each module is configured to provide a skin or body treatment, underlying tissue treatment, or both. One or more applicators are coupled with the skin or body treatment device. Each applicator enables the use of the one or more removable modules to effect the skin or body or underlying tissue treatment. A selected skin or body treatment, underlying tissue treatment, or both can be implemented and performed upon selection and configuration of the one or more removable and replaceable modules and the one or more applicators required for the selected treatment.

### RELATED APPLICATION

This application claims priority to, and the benefit of, co-pending U.S. Provisional Application No. 60/964,246, filed Aug. 10, 2007, for all subject matter common to both applications. The disclosure of said provisional application is hereby incorporated herein by reference in its entirety.

### FIELD OF THE INVENTION

The present invention relates to a system for treating a physical skin or body condition, and more particularly relates to a multi-module treatment system and corresponding method of use for various skin or underlying tissue treatments, including reducing cellulite, girth reduction, and aging effects.

### BACKGROUND OF THE INVENTION

Individuals presently undergo non-invasive or minimally invasive skin or body treatment procedures to address problems such as cellulite, wrinkles, blotches, sun spots, acne, discolorations, scars, pigmentation, acne, rosacea, actinic keratosis, telangiectasia, sun/photo-damage, unwanted hair, varicose vein or other skin conditions. These non-invasive and/or minimally invasive procedures are performed, most of the time, without the need of an anesthetic, and the patients do not have “down-time” after the procedures. The procedures last from a couple of minutes to a couple of hours, depending on the treatment type and the size of the area to be treated.

With the recent advances in the technology, it is possible to apply a vast variety of treatment techniques as non-invasive procedures. These techniques may use a laser beam, light emitting diodes (LED), ultrasound vibes, radio-frequency (RF) energy, electrical stimulation, acoustic shock, and/or intense pulsed light (IPL). Even though self-applicator kits can be found in the market using some of these technologies, these kits are less effective because the power source that they use is weaker than those found on devices available to a licensed technician or under a doctor's supervision. Furthermore, when using these technologies, especially the laser treatment, it is imperative that a certified technician is present and controls the procedure. Even though these procedures are not harmful when performed correctly, if applied incorrectly, they may cause permanent skin damage.

Skin or body treatments are often performed at locations such as spas, clinics or other certified treatment centers. Currently, these centers have separate machines for each treatment type, resulting in a treatment machine clutter at the treatment center. As such, they can only use one type of treatment on one patient at a given time using one machine.

### SUMMARY

There is a need for a multi-module skin or body treatment device giving a user or practitioner the flexibility to select certain skin or body treatment devices and manage one or more devices from a central location. Such a single skin or body treatment device providing a plurality of treatment types eliminates the need to have multiple skin or body treatment devices and results in an effective way of applying multiple treatments to one or more patients. Furthermore, such a device enables the combination of one or more types of treatments generating synergistic effects and increasing the overall treatment effectiveness, in some cases unexpectedly. The present invention is directed toward further solutions to address this need, in addition to having other desirable characteristics.

In accordance with one example embodiment, a skin or body treatment device includes one or more removable and replaceable modules coupled with the skin or body treatment device. Each module is configured to provide a skin, body, or underlying tissue treatment. One or more applicators are coupled with the skin or body treatment device. Each applicator enables the use of the one or more removable modules to effect the skin, body, or underlying tissue treatment.

In accordance with aspects of the present invention, the one or more removable and replaceable modules can be a laser module, a light emitting diode module, an ultrasound module, a massage module, an intense pulsed light module, a microdermabrasion module, an electrical stimulation module, a radio frequency module, acoustic shock module, or any combination thereof. The skin or body treatment device can be configured for use of a plurality of the one or more removable and replaceable modules at one time. The skin or body treatment device can be configured for use of one of the one or more removable and replaceable modules by a plurality of the one or more applicators at one time.

In accordance with further aspects of the present invention, the skin or body treatment device can further include an input device connected to the skin or body treatment device, wherein a practitioner enters treatment specific parameters using the input device. One or more output devices can be connected to the skin or body treatment device, wherein the one or more output devices display results of one or more treatments.

In accordance with further aspects of the present invention, the one or more applicators are removable and replaceable. A data storage device can be provided, as well as a treatment module repository. Likewise a processor can be provided for controlling the one or more removable and replaceable modules. The processor can also or alternatively manage and implement data storage.

In accordance with aspects of the present invention, the method can further include providing patient specific parameters to the multi-module skin or body treatment device. The multi-module skin or body treatment device can determine a desired course of treatment and communicating the course of treatment based on the patient specific parameters.

In accordance with further aspects of the present invention, the skin or body treatment device can include a diagnostic capability. A diagnostic software application can receive patient symptom information, process the information, and output a diagnosis. In addition, the skin or body treatment device can further output a treatment regimen in view of the diagnosis.

### BRIEF DESCRIPTION OF THE DRAWINGS

The aforementioned features and advantages, and other features and aspects of the present invention, will become better understood with regard to the following description and accompanying drawings, wherein:

FIG. 1 is a diagrammatic illustration of a skin or body treatment system according to one embodiment of the present invention;

FIG. 2 is a diagrammatic illustration of a treatment module repository according to one embodiment of the present invention;

FIG. 3 is a diagrammatic illustration of a skin or body treatment device according to one embodiment of the present invention;

FIG. 4A is a diagrammatic illustration of the skin or body treatment device with multiple treatment modules and multiple patients according to one embodiment of the present invention;

FIG. 4B is a diagrammatic illustration of the skin or body treatment device with one treatment module and multiple patients according to one embodiment of the present invention;

FIG. 4C is a diagrammatic illustration of the skin or body treatment device with multiple treatment modules and one patient according to one embodiment of the present invention;

FIG. 4D is a diagrammatic illustration of the skin or body treatment device with multiple output devices according to one embodiment of the present invention;

FIG. 4E is a diagrammatic illustration of the skin or body treatment device with one output device according to one embodiment of the present invention; and

FIG. 5 is a flowchart illustrating a method of using the skin or body treatment device according to one embodiment of the present invention.

### DETAILED DESCRIPTION OF THE INVENTION

The present invention generally relates to a multi-module skin or body treatment system. The skin or body treatment system of the present invention may apply electromagnetic radiation, electrical stimulation, light stimulation, ultrasound emission, acoustic shock, abrasion techniques and/or massage to an area targeted for, e.g., cellulite and girth reduction or anti-aging procedures, in one or more predetermined wavelengths, intervals, periods, and/or patterns. Other tissue treatments are possible with variation of the electromagnetic radiation, electrical stimulation, ultrasound emission, abrasion techniques and/or massage applications. The modular aspect of the inventive system enables a practitioner to select the treatments to be provided and to quickly and easily add or remove available treatments to the overall system by adding or removing modular components from the system that are removable and replaceable.

The multi-module skin or body treatment system described herein may include one or more of the following modules: a laser module, a light emitting diode (LED) bio-stimulation module, an ultrasound module, an acoustic shock module, a massage module, a radio frequency (RF) module, an electrical stimulation module, a microdermabrasion module, an intense pulsed light (IPL) module, and the like. An ordinarily skilled artisan would appreciate that the modules are not limited to foregoing modules, and that additional or other combinations of modules may be used with the multi-module skin or body treatment system described herein.

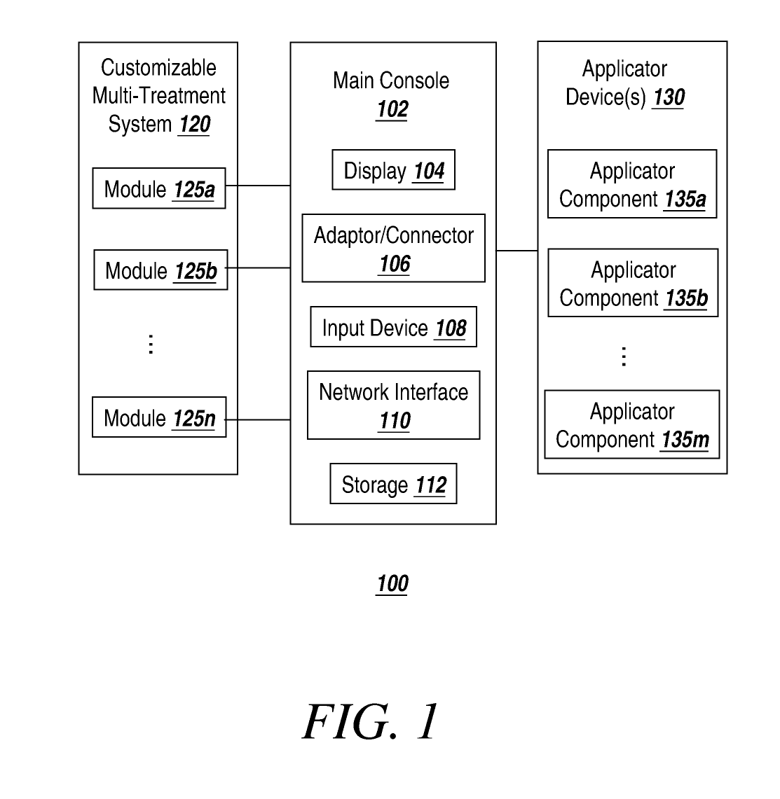

Referring now in detail to the drawings wherein like parts are designated by like reference numerals throughout, FIG. 1 illustrates a system for the treatment of a condition, and preferably a tissue, according to the present invention. Although the present invention will be described with reference to the example embodiments illustrated in the figures, it should be understood that the present invention can be embodied in many alternative forms. In addition, any suitable size, shape, or type of elements or materials can be utilized, and can be employed to treat the various conditions set forth above. The terms skin and tissue are used alternatively throughout the application bearing a similar meaning. One ordinarily skilled artisan will recognize that in any exemplary embodiment described herein, either skin or tissue could be employed and the utilization of one term over another is not limiting.

According to one aspect of the present invention an exemplary skin or body treatment system 100 is provided. The skin or body treatment system 100 includes a main console 102, a customizable multi-treatment system 120, and one or more applicator devices 130. The customizable multi-treatment system 120 has multiple modules such as modules 125a-125n that can be connected to the main console 102 via one or more adaptor/connectors 106. Each module provides a specific skin or body treatment technology or a combination of skin or body treatment technologies, including, but not limited to, ultrasound, acoustic shock, electrical stimulation, radio frequency (RF), intense pulse light source (IPL), light emitting diode (LED) bio-stimulation, laser, massage therapy, and/or microdermabrasion. In one embodiment, each module in the multi-treatment system 120 is fixedly connected to the main console 102. In another embodiment, each module 125 in the multi-treatment system 120 is a removable module that can be disconnected from the main console 102. As such, only the modules that are needed for treatment of specific patient(s) are required to be connected to the main console 102. In addition, if an upgraded module is available or if an existing module is broken, a physician can purchase a new or upgraded module without having to replace the entire skin or body treatment system 100 or the entire customizable multi-treatment system 120. Furthermore, a physician or certified professional can decide to purchase only the modules that are suitable to be used with his/her practice.

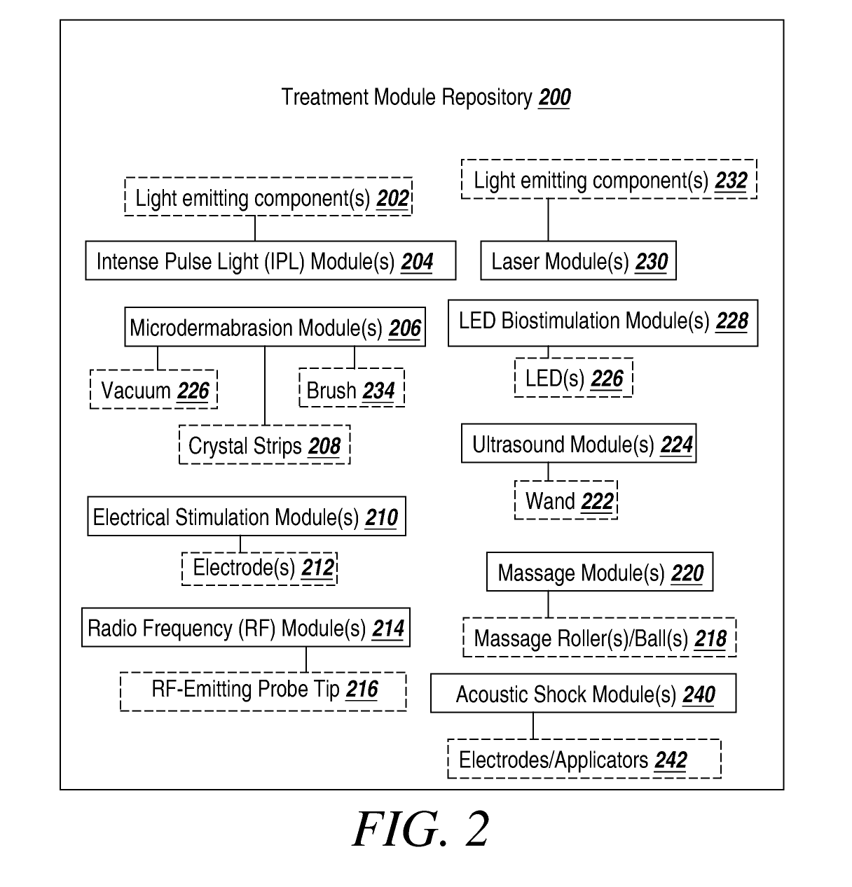

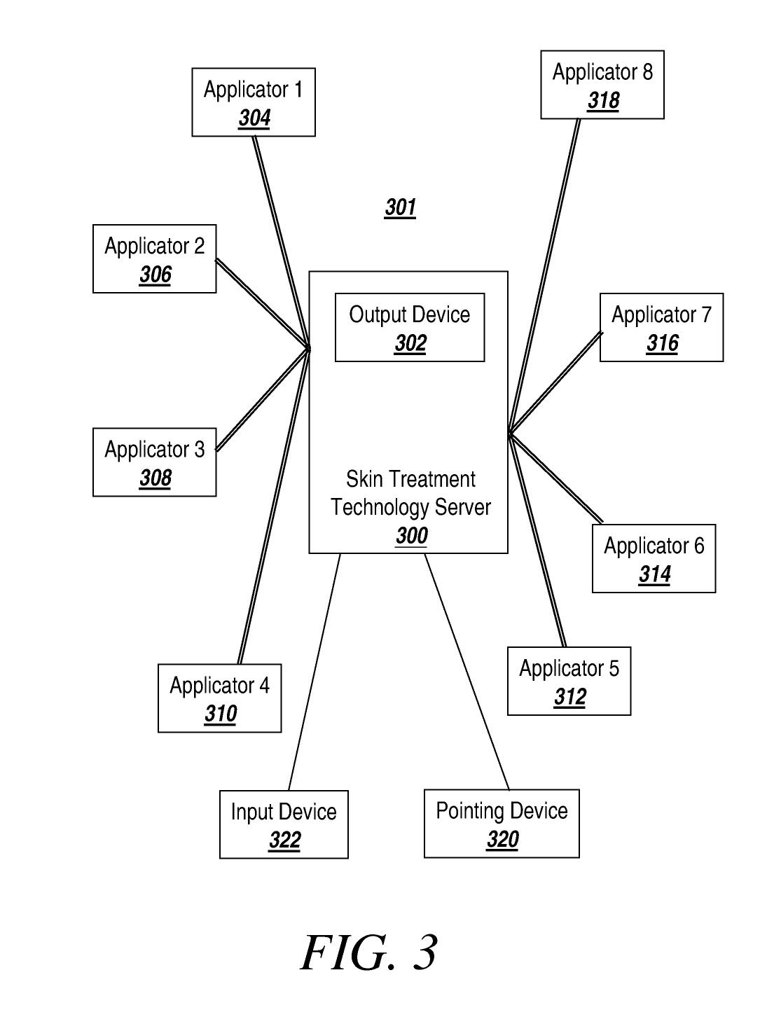

Each module in the customizable multi-treatment system 120 can have one or more corresponding applicator components 135 that are to be used with one or more applicator devices 130. In one embodiment, applicator components 135a-135m corresponding to different modules can be used in a single applicator device 130. In another embodiment, applicator components 135a-135m for different modules can be used in different applicator devices 130. The applicator components 135a-135m may be an ultrasound sound-head, a wand with LED tip, a wand with laser-emitting tip, a massage device, a wand with an RF-emitting tip, a pair of electrodes, a wand with a brush and means to apply crystals or a wand with a light-emitting tip. Different types of applicator components are illustrated in FIGS. 2-3 and will be described below.

The main console 102 allows a user to view menus and results using the various skin or body treatment modules on display 104. The main console 102 has an input device 108 that allows a user to input commands and data to the main console 102. The input may include required parameters for the type of treatment to be performed. Patient's information and/or physician's experience can be used to customize a treatment for the patient. The treatment depends on the patient's condition, thus different treatments may be required for different conditions. For example, a modulated energy source is better suited at certain times, while at other times, a pulse energy source may be better suited. Data including patient's information and treatment customizations may be stored in a storage component 112. Alternatively, data may be stored on a database accessible via a network interface 110. Network interface 110 is able to interface to a Local Area Network (LAN), Wide Area Network (WAN) or the Internet through a variety of connection types including, but not limited to, standard telephone lines, LAN or WAN links (e.g., 802.11, T1, T3, 56 kb, X.25), broadband connections (e.g., Integrated Services Digital Network (ISDN), Frame Relay, asynchronous transfer mode (ATM)), wireless connections, etc., or some combination of any or all of the above. Network interface 110 may include a built-in network adapter, network interface card (NIC), Personal Computer Memory Card International Association (PCMCIA) network card, card bus network adapter, wireless network adapter, Universal Serial Bus (USB) network adapter, modem or any other device suitable for interfacing the main console 102 to any type of network capable of communication and performing operations described herein.

In addition, the skin or body treatment system 100 can further include a diagnostic capability. For example, the skin or body treatment system 100 can incorporate a diagnostic application that requests information from a user, processes that information, and then returns a diagnosis and recommended treatment regimen.

FIG. 2 is a diagrammatic illustration of an exemplary treatment module repository 200 that is suitable for use in conjunction with the skin or body treatment system illustrated in FIG. 1. A treatment module may be selected from the treatment module repository 200 to be used in a customizable multi-treatment system 120. A treatment module repository 200 may be provided with a skin or body treatment system 100 to store modules that are not currently used in the customizable multi-treatment system 120. The treatment module repository 200 can alternatively or in addition support active use of the various modules. Further, the treatment module repository 200 can be separate from the main console 102, or integral therewith.

Treatment module repository 200 may include one or more laser modules 230. A laser module 230 can, if directed, emit radiation at particular wavelengths. The laser module 230 emits coherent, monochromatic light, in a stimulated emission. The laser module 230 can project radiation having wavelengths in a concentric combination of infrared and visible laser light, for example between about 650 nanometers and about 1295 nanometers. In accordance with an example implementation, the device can project radiation at about 650 nanometers and 915 nanometers; or 650 nm, 915 nm, and 1210 nm; or 650 nm and 1210 nm. In one embodiment, the laser module 230 may be a fixed power laser, such that the power only varies with the exposure time; i.e., increase exposure time to increase the power. Alternatively, the laser module 230 may have variable power outputs, or different power levels, that can be employed in accordance with the teachings of the present invention.

A laser module 230 may include one or more light emitting components 232. In a preferred embodiment, the one or more light emitting components 232 are replaceable components, such that the entire laser module 230 does not need to be replaced if the light emitting component 232 is broken.

Treatment module repository 200 includes one or more LED bio-stimulation modules 228. Bio-stimulation is reactivation of cell functions to restart the regenerative processes. Photonic energy (light) produced by the laser or LED is absorbed by the skin and cells of the body, the photonic energy is converted into chemical energy which initiates a cascade of events at a cellular level. Affected cells optimize their function and improve their cell communication capability. Bio-stimulation increases the amount of Adenosine Tri-Phosphate (ATP) production and increases the cellular activity. Each LED bio-stimulation module 228 includes one or more LEDs 226. In a preferred embodiment, the LEDs 226 are replaceable components such that if any of the LEDs 226 is malfunctioning, only the malfunctioning LED needs to be replaced. LEDs may be of different colors, where different colors may be used for different types of treatments. Bio-stimulation may be applied to a patient using replaceable hand-held LED pads. There can be a different replaceable pad for each LED color. Alternatively, the replaceable pads may be configured to accept different LED color sockets. This way, the life of the pads and the LED bio-stimulation module may be further increased by replacing the burn-out LED color sockets instead of replacing the hand-held pads.

The treatment repository 200 may include one or more ultrasound modules 224. Each ultrasound module 224 includes one or more wands 222. In a preferred embodiment, each wand 222 is replaceable without replacing the entire ultrasound module 224. Ultrasonic sound waves are at a frequency of more than 20,000 Hz and are not heard by the human ear, In the ultrasound treatment, duty cycles less than 100% are usually termed pulsed ultrasound, while a 100% duty cycle is referred to as continuous ultrasound. Ultrasonic sound waves stimulate body cells, causing movement of cytoplasm, the rotation of mitochondria, and the vibration of the cell nucleus. As a result they stimulate and expand the cell membrane. Ultrasound treatment improves local blood and lymph circulation. Ultrasonic treatment further helps to improve the metabolism and regeneration of body cells.

Ultrasound treatments initiate sonophoresis that uses ultrasonic frequencies to help transfer substances of high molecular weight throughout the skin. Low-frequency ultrasound just above the range that is audible to the human ear enables dissolved gases in liquid regions between skin cells to bubble and move, clearing toxins caused by free radical activity. The sonophoretic action creates pathways into the skin, allowing specific molecules to enter. Sonophoresis is done using low-frequency ultrasound—sound waves oscillating at a rate above 20 kilohertz.

Additionally, focused ultrasound at frequencies of 5-15 Mz produce thermal and/or mechanical effects deep within the tissue, including but not limited to heating and coagulation similar to the changes induced by the ablative and non-ablative laser and light sources.

The treatment repository 200 may further include one or more massage modules 220. A massage module 200 may include one or more massage rollers or balls 218. In a preferred embodiment, each massage roller or ball 218 is replaceable without replacing the entire massage module 218. The process of skin or body massage results in friction and heat generation, which may limit the treatments due to pain. The process is also aimed at mobilizing trapped fluid. A subjective feeling of cold caused by the metal material forming the rollers can enable application of a more aggressive massage treatment because of the reduced pain perceived by the patient during treatment. In addition, the cool surface of the massage rollers or balls 218 when touching the skin or body facilitates lymphatic circulation by creating a temperature differential at the skin/roller interface. This promotes a pumping action that allows trapped fluid to clear the skin and thereby reduce the appearance of bumps and cellulite.

Massaging of the exposed skin surface can occur using a mechanical massage mechanism. Massaging the exposed skin can be executed by massaging from points distal from a heart of the patient to points proximal to the heart of the patient to encourage fluid flow toward the heart. Further, massaging the exposed skin can include massaging in predetermined patterns of motion.

The treatment repository 200 may also include one or more radio frequency (RF) modules 214. An RF module 214 may include one or more RF-emitting probe tips 216. In a preferred embodiment, each RF-emitting probe tip 216 or a pair of probe tips 216 are replaceable without replacing the entire RF module 214. The probe tips 216 may have different end shapes and may be of different dimensions. Appropriate probe tip 216 may be chosen according to the needs of the procedure. For example, the facial area may require RF-emitting probe tips 216 with smaller contact area to increase precision and decrease discomfort at each application, whereas larger areas with thicker skin may require RF-emitting probe tips 216 with a larger contact area.

The RF module 214 may use a vacuum to fold the skin, or to simply pull up on the skin, to ensure tight contact and position the dermis in direct alignment with the path of radio frequency. The combination of this vacuum positioning and the use of topical conductive coupling medium may concentrate heat deep in the dermis, leading to collagen contraction and regeneration. Energy from a RF generator is delivered to the RF-emitting probe tip 216, which may be equipped with a cooling spray, computer chip and sophisticated sensors to measure pressure current flow and skin temperature. Feedback from the RF-emitting probe tip 216 to the multi-module skin or body treatment device may regulate the amount of energy applied to the tissue. When the unit is fired, the RF-emitting probe tip 216 is cooled, followed a moment later with current flow to the deep layers of the skin by capacitive coupling. Cooling continues for an additional second or two. The patient feels a sensation of cold, then heat, then cold again. The combination of capacitive coupling and aggressive cooling protects the epidermis during the delivery of energy to the deep dermis and subcutaneous layers. Collagen heating produces microscopic wounding, with immediate collagen contraction, and subsequent repair and remodeling of this deep collagen produces skin or body tightening over time.

The treatment repository 200 may further include one or more electrical stimulation modules 210. An electrical stimulation module 210 may include one or more electrodes 212. In a preferred embodiment, each individual electrode 212 is replaceable without replacing the electrical stimulation module 210. It is also preferred that the electrodes 212 may be used with more than one electrical stimulation module 210 such that if the electrical stimulation module 210 needs to be replaced or upgraded, the electrodes 212 may be reused.

Electrical stimulation is defined as the use of an electrical current to transfer energy to the skin or body. The type of electricity that is transferred is controlled by the electrical source. Capacitatively coupled electrical stimulation involves the transfer of electric current through an applied surface electrode 212 that is in wet (electrolytic) contact with the external skin surface. When capacitatively coupled electrical stimulation is used, two electrodes 212 may be required to complete the electric circuit. Electrodes 212 may be placed over a wet conductive medium on the skin.

Electrical stimulation may be applied in one of two ways. For the first method, one electrode 212 (positive or negative polarity) is applied to sterile, conductive material, such as saline-moistened gauze, placed on the skin. The conductive surface of the other electrode 212 is applied nearby on intact dry skin. The second method involves positioning the conductive surfaces of two electrodes 212 with the same polarity on intact dry skin on opposite borders of the are to be treated, straddling the treatment area, e.g. a wrinkle. A third electrode 212 with the opposite polarity is placed nearby on intact dry skin. In both methods, the pulse frequency may be set to 100 pulses/second and the voltage may be set to deliver a current that produces a moderately strong but comfortable tingling sensation. The voltage required is typically between 50 and 150 volts. Polarity of one or more electrodes 212 placed on or straddling the treatment area depends on the treatment needs.

Treatment module repository 200 may also include one or more microdermabrasion modules 206. A microdermabrasion module 206 may include one or more crystal strips 208 that are disposable. The crystal strips 208 may be coupled with a brush 234 to brush off the crystals sprayed on the tissue. Alternatively, the crystal strips 208 may be coupled with a vacuum 236 that vacuums off the crystals sprayed in the tissue. As such, a physician may decide to provide patients with their own crystal strips 208 to be used with the microdermabrasion module 206. Alternatively, a physician may decide to use new crystal strips 208 for each treatment. In microdermabrasion, the dead outermost surface of the skin is partially or completely removed by light abrasion. Different methods include mechanical abrasion from jets of zinc oxide or aluminum oxide crystals, fine organic particles, or a roughened surface. Particles and removed material are brushed off through the wand/handpiece 208 through which the abrasive particles come. Microdermabrasion may be used to remove sun-damaged skin and to remove or lessen scars and dark spots on the skin.

Treatment module repository 200 may further include one or more intense pulse light (IPL) modules 204. Each intense pulse light (IPL) module 204 includes one or more light emitting component(s) 202. The one or more light emitting component(s) 202 may be a replaceable component such that the entire IPL module 204 does not need to be replaced if one of the light emitting components 202 is broken. The IPL module 204 may use “cut off” filters to selectively deliver the desired wavelengths. These wavelengths can be customized to reach the specific hair, blood vessels, or skin or body component being treated, and can be modified with each pulse. IPL begins with all wavelengths of light from 500 to 1200 nanometers (nm)—including green, yellow, red and infrared light. Various lower range cut off filters (ranging from 515-755) block light shorter than the wavelength of the cut off filter. During treatment, IPL may target melanin in sunspots, melasma and hair follicles, and the hemoglobin in blood vessels.

Treatment module repository 200 may further include one or more acoustic shock modules 240 coupled with electrodes/applicators 242 for administering the acoustic shock therapy. Acoustic shock waves have a number of different applications including breaking up concretions in the human body and to treat numerous conditions including those related to soft-tissue, nerves, and bone. In general, acoustic shock works through the implementation of an electrical spark discharge generated between two electrodes in a fluid. The spark discharge causes shock waves to form in the fluid. A reflector focuses the shock waves toward an area being treated. A membrane can be used to couple the waves traveling through the fluid with the human body through physical contact.

The tissue treatment system, and corresponding method of use, can be effective in treating a selected condition, such as unwanted tissue, edema, overactive sweat glands, cysts, lipomas, psoriatic tissue, acne, excess lymphatic fluid and tissue, varicose veins, telengiectasias, pain, inflammation, inflammatory products, and body toxins.

Now referring to FIG. 3, a skin or body treatment device according to one embodiment of the present invention will be described. The skin or body treatment device 301 may have a skin or body treatment technology server 300, an input device 322, a pointing device 320 and one or more applicators 304-318. The skin or body treatment technology server 300 can be a computer or CPU, or other hub or control center, or central organizing device, that can manage the connections with one or more modules and applicators and potentially coordinate the application of the various skin or body treatments, or convey communications from other portions of the system. Accordingly, the applicators 304-318 may be controlled at the same time by the skin or body treatment technology center 300. The applicators may be one of an ultrasound sound-head, a wand with LED tip, a wand with laser-emitting tip, a massage device, a wand with RF-emitting tip, a pair of electrodes, a wand with brush and means to apply crystals or a wand with light-emitting tip. If the treatment requires parameters, such as, weight, skin tone, treatment duration, treatment intensity or skin type, the parameters may be entered to the skin or body treatment technology server using an input device 322. A pointing device 320, such as a mouse or a pointer, may be used to enter parameters. Alternatively, the skin or body treatment technology server 300 may have a touch-screen to enter/select the necessary parameters and/or information. An output device 302, such as a screen, may be used to view the results of the treatment. The output device 302 may also be used to fine-tune the treatment parameters. The skin or body treatment device 301 enables certified practitioners to treat multiple patients at the same time. Alternatively, the skin or body treatment device 301 may be used to apply different treatments to a single patient.

In addition, the treatment technology server 300 can further include a diagnostic capability in accordance with one example embodiment of the present invention. Specifically, a diagnostic software application can operate on the treatment technology server 300. The input device 322 can provide prompts for a user to input information relating to symptoms of a patient. The diagnostic software can then process that information and return a diagnosis and recommended treatment regimen. Example software applications include a treatment input routine for capturing the treatment condition and treatment parameters. Inputs from the treatment input routine will be sent to the initial settings function to determine a number of treatment module options. The treatment module options will be displayed on the input device. The operator will select the appropriate module, perform a test on the patient and enter the response into the input device. The response data will be used by the test case input function to determine the final treatment module settings. The final settings routine will display the final treatment module recommendations.

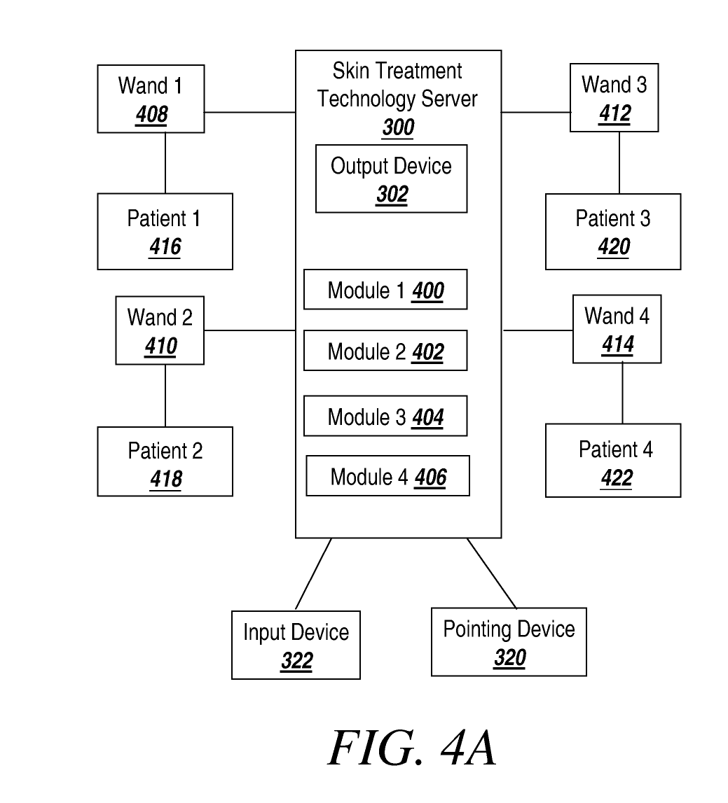

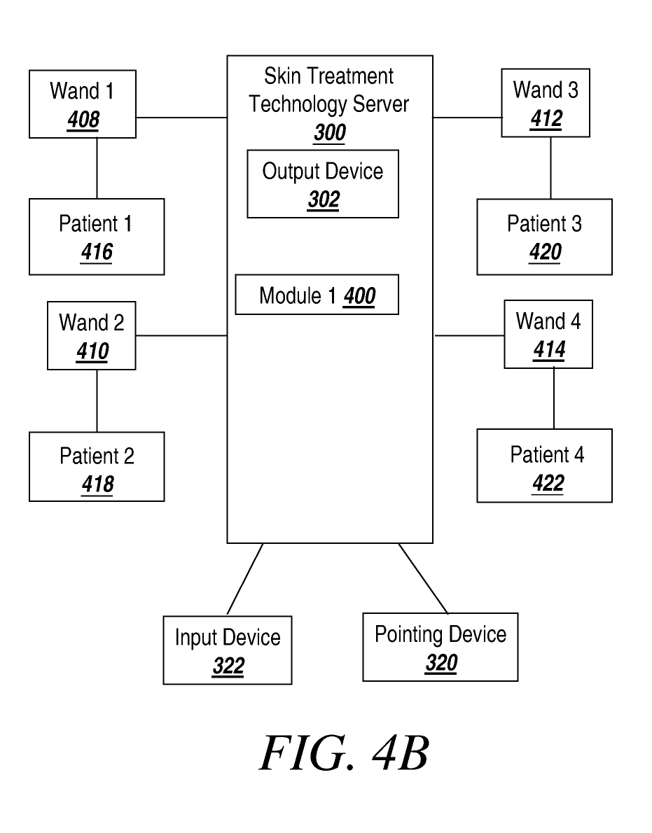

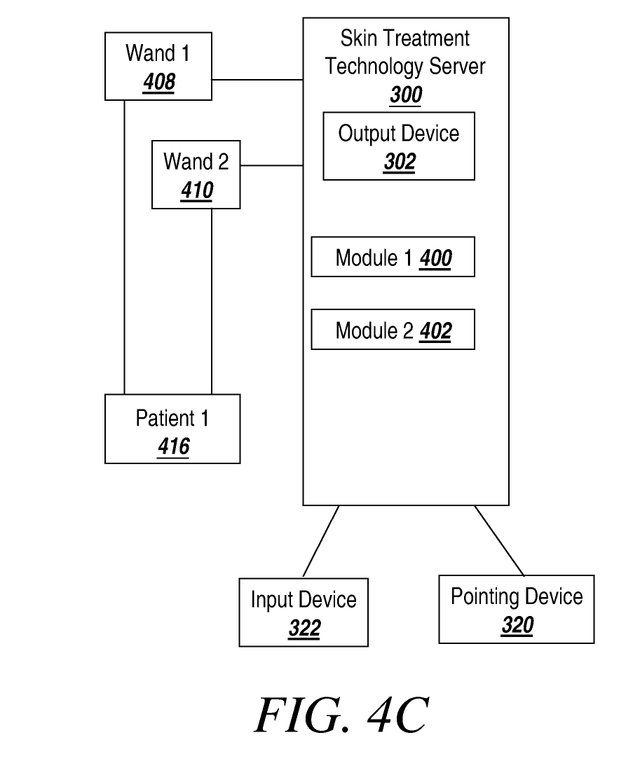

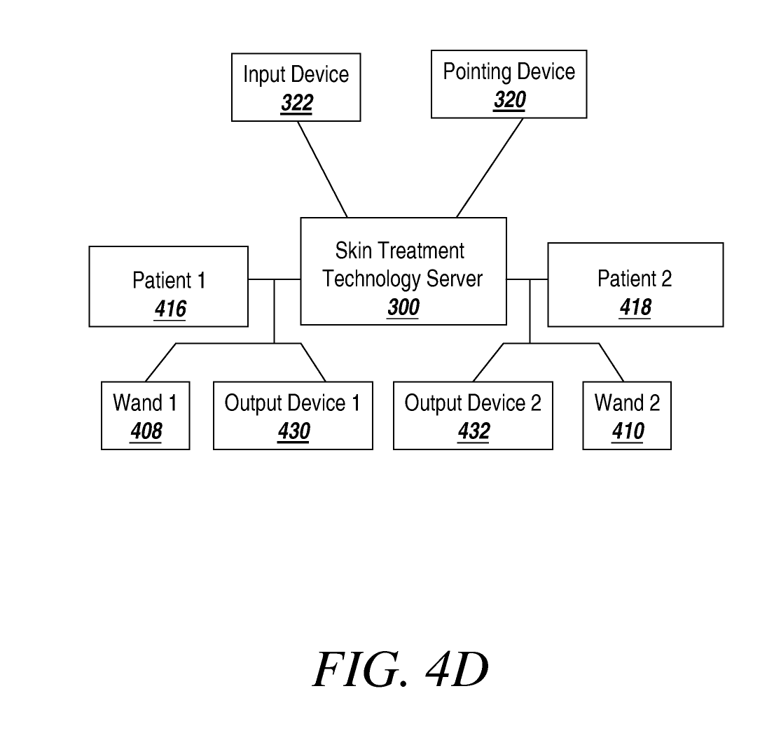

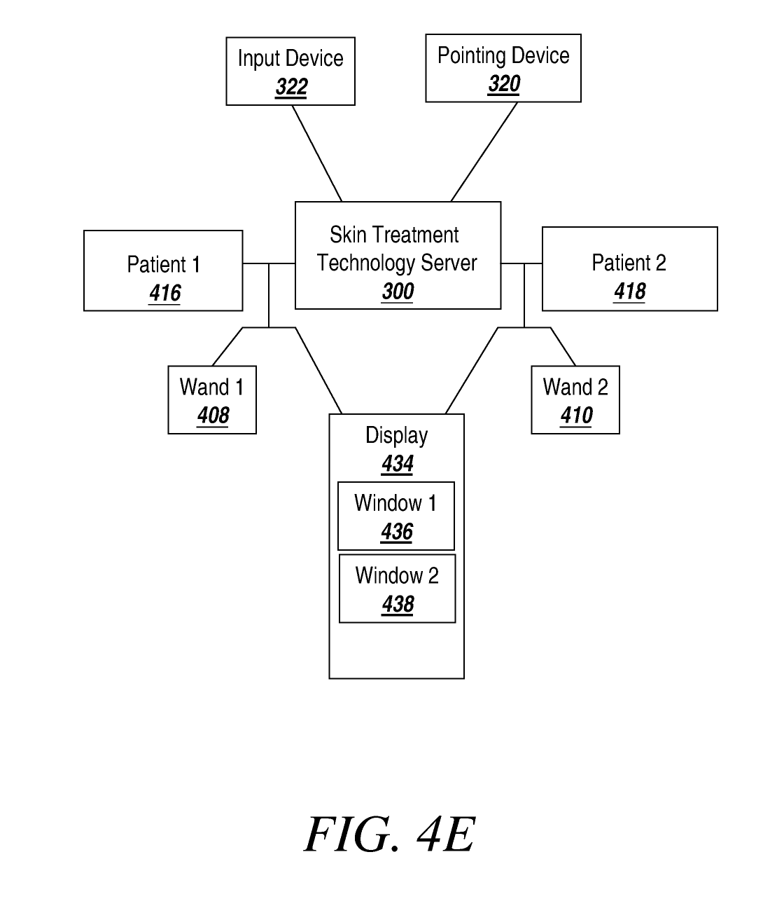

FIG. 4A illustrates the skin or body treatment device 301 being used to treat four different patients 446-422. As illustrated in this example, four treatment modules 400-406 are connected to the skin or body treatment technology server 300. The treatment modules 400-406 may be modules for different types of treatments, or they all may be modules for the same type of treatment. Each patient 416-422 is treated using a different applicator or wand 408-414. Patients may bring their own personal wands that are compatible with the skin or body treatment technology server 300. Alternatively, patients may use the wands that are available to them at the treatment center. Each patient may use one or more treatment options provided by the modules 400-406. For example patient-one 416 may get a microdermabrasion treatment using wand 408, while patient-two 410 may get a LED bio-stimulation treatment using wand 410. One ordinarily skilled artisan would appreciate that the combinations are not limited to the ones described here, and other treatment combinations may be used as needed.

FIG. 4B illustrates another example where the skin or body treatment device 301 is used for four different patients 416, 418, 420, and 422 with a single treatment module 400. It is possible to apply the same treatment option provided by module 400 to a plurality of patients 416-422. In this example, the wands 408, 410, 412, and 414 provide the same treatment, specifically the treatment option provided by module 400. However, the specifications of the wands 408-414 may differ according to the parameters specific to each patient 416-422. For example, module 400 may provide the LED bio-stimulation treatment option. However, wands 408-414 may have different color LEDs or may emit lights at different intensities according to the specifications of each patient 416-422. The treatment time for each patient 416-422 may be different.

FIG. 4C illustrates one exemplary embodiment where a single patient 416 receives two types of treatments provided by two modules 400-402 using two wands 408-410. An ordinarily skilled artisan would appreciate that the treatment number is not limited to two treatments, and the use of two treatments should be construed as exemplary and not limiting. Module 400 and module 402 may provide different treatment options. Alternatively, module 400 and module 402 may provide the same treatment applied to different parts of the body using wand 408 and wand 410. In this embodiment, the patient receives multiple treatments at the same time, hence the patient saves time. Multiple treatments must be assessed by a certified professional to decide on the pain and discomfort that they may cause on the patient. The certified professional or practitioner should also assess whether any two or more treatments should not be combined due to possible adverse effects.

The results of the treatments may be viewed on an output device 302, such as a screen or a printer to print the results on a paper. As illustrated in FIG. 4D there may be multiple displays 430-410 provided for each patient 416-418, respectively. Alternatively, as illustrated in FIG. 4F, the results may be viewed on a single output device 434, providing different windows 436-438 for each patient 416-418, respectively.

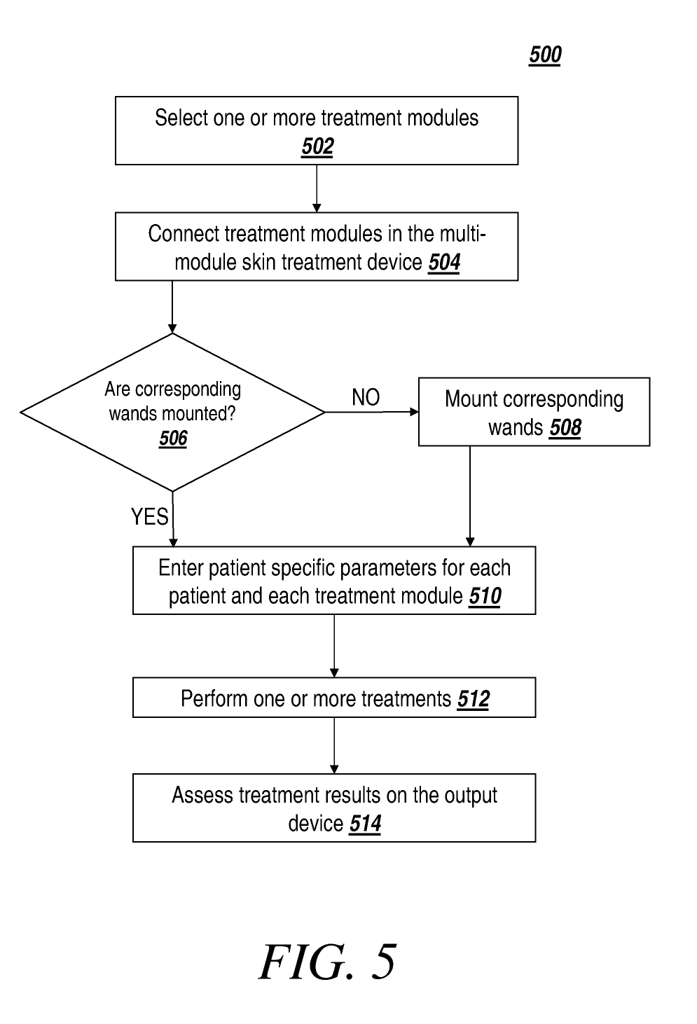

FIG. 5 is a flowchart 500 describing a method of using the multi-module skin or body treatment system described above. A certified practitioner or clinician selects the required module(s) corresponding to the treatment type(s) chosen by the patient, (step 502). The practitioner then connects the modules to the multi-module skin or body treatment device (step 504). The wands or applicators associated with the module may be already mounted on the multi-module skin or body treatment device, (step 506). If not, the practitioner mounts the necessary wands or applicators corresponding to the selected treatment(s), (step 508). The practitioner then enters patient specific parameters for each patient and for each treatment to the multi-module skin treatment device, (step 510). The practitioner performs the selected treatment(s), (step 512), and assesses the treatment results as they are displayed on the output device, (step 514).

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention, and exclusive use of all modifications that come within the scope of the appended claims is reserved.

What is claimed is:

1. A skin or body treatment device comprising:
one or more removable and replaceable modules coupled with the skin or body treatment device, wherein each module is configured to provide a skin or body treatment, an underlying tissue treatment, or both; and
one or more applicators coupled with the skin or body treatment device, wherein each applicator enables the use of the one or more removable modules to effect the skin or body treatment, underlying tissue treatment, or both.

2. The skin or body treatment device of claim 1, wherein the one or more removable and replaceable modules comprise a laser module, a light emitting diode module, an ultrasound module, an acoustic shock module, a massage module, an intense pulsed light module, a microdermabrasion module, an electrical stimulation module, a radio frequency module, or any combination thereof.

3. The skin or body treatment device of claim 1, wherein the skin or body treatment device is configured for use of a plurality of the one or more removable and replaceable modules at one time.

4. The skin or body treatment device of claim 1, wherein the skin or body treatment device is configured for use of one of the one or more removable and replaceable modules by a plurality of the one or more applicators at one time.

5. The skin or body treatment device of claim 1, further comprising:
an input device connected to the skin or body treatment device, wherein a practitioner enters treatment specific parameters using the input device.

6. The skin or body treatment device of claim 1, further comprising:
one or more output devices connected to the skin or body treatment device, wherein the one or more output devices display results of one or more treatments.

7. The skin or body treatment device of claim 1, wherein the one or more applicators are removable and replaceable.

8. The skin or body treatment device of claim 1, further comprising a data storage device.

9. The skin or body treatment device of claim 1, further comprising a treatment module repository.

10. The skin or body treatment device of claim 1, further comprising a processor for controlling the one or more removable and replaceable modules.

11. The skin or body treatment device of claim 1, further comprising a processor for managing and implementing data storage.

12. The skin or body treatment device of claim 1, further comprising a diagnostic software application operating on the device for receiving input and outputting a diagnosis.

13. The skin or body treatment device of claim 1, further comprising a diagnostic software application configured to receive input from a user and output a diagnosis and a treatment regimen.

14. A method of using a multi-module skin or body treatment system, comprising:
selecting one or more modules corresponding to desired skin or body treatments, underlying tissue treatments, or both;
configuring the one or more modules for use;
configuring one or more applicators corresponding to the one or more required modules for use;
initiating and operating the one or more modules to effect a skin or body treatment, underlying tissue treatment, or both.

15. The method of claim 14, further comprising providing patient specific parameters to the multi-module skin or body treatment device.

16. The method of claim 15, further comprising the multi-module skin or body treatment device determining a desired course of treatment and communicating the course of treatment based on the patient specific parameters.

17. The method of claim 14, wherein the one or more modules comprise a laser module, a light emitting diode module, an ultrasound module, an acoustic shock module, a massage module, an intense pulsed light module, a microdermabrasion module, an electrical stimulation module, a radio frequency module, or any combination thereof.

18. The method of claim 14, further comprising displaying results of the skin or body treatment, underlying tissue treatment, or both.

19. The method of claim 14, further comprising a processor controlling the one or more modules during operation.

20. The method of claim 14, further comprising storing data relating to operation of the multi-module skin or body treatment system and to a specific patient.

21. The method of claim 14, further comprising inputting patient symptom information into the skin or body treatment system and the system outputting diagnostic information.

22. A skin or body treatment device comprising:
one or more removable and replaceable modules coupled with the skin or body treatment device, wherein each module is configured to provide a skin or body or underlying tissue treatment; and
one or more applicators coupled with the skin or body treatment device, wherein each applicator enables the use of the one or more removable modules to effect the skin or body or underlying tissue treatment;
wherein the one or more removable and replaceable modules comprise a laser module, a light emitting diode module, an ultrasound module, a massage module, an intense pulsed light module, a microdermabrasion module, an electrical stimulation module, a radio frequency module, or any combination thereof.

23. The skin or body treatment device of claim 22, wherein the one or more removable and replaceable modules and the one or more applicators are selectable based on a desired skin or body treatment to be performed.
